(19) United States
(12) Patent Application Publication
(10) Pub. No.: US 2026/0232924 A1
(43) Pub. Date: Aug. 13, 2026

(54) HYDROGEL DELIVERY ACTUATORS WITH DUAL-AXIS NEEDLE POSITIONING

(71) Applicants: BOSTON SCIENTIFIC SCIMED, INC., Maple Grove, MN (US); BOSTON SCIENTIFIC MEDICAL DEVICE LIMITED, Galway (IE)

(72) Inventors: Sushil Kumar, Gurgaon (IN); Amit Kumar, Noida (IN); Priyanshu Gupta, Gurgaon (IN); Umesh Kumar Badaya, Bandikui (IN); Jonathan J. Lund, Glencoe, MN (US); Patrick S. Nicolas, Mansfield, MA (US)

(73) Assignees: BOSTON SCIENTIFIC SCIMED, INC., Maple Grove, MN (US); BOSTON SCIENTIFIC MEDICAL DEVICE LIMITED, Galway (IE)

(21) Appl. No.: 19/534,374

(22) Filed: Feb. 9, 2026

Related U.S. Application Data
(60) Provisional application No. 63/756,619, filed on Feb. 10, 2025.

(51) Int. Cl. A61M 5/32; A61M 5/315

(52) U.S. Cl. CPC A61M 5/326; A61M 5/31501; A61M 5/31511; A61M 5/31513

(57) ABSTRACT

In some aspects, the disclosure provides a hydrogel deployment actuator comprising a housing having an ergonomic hand grip, a syringe tray configured to be movably disposed in the housing and receive and secure a syringe, a dual-axis swivel head assembly comprising a dual-axis swivel head, a vertical swivel knob, a horizontal swivel knob, a swivel lock mechanism configured to lock both vertical and horizontal movement of the dual-axis swivel head, and a flexible tube configured to fluidically couple the dual-axis swivel head to a proximal end of the syringe, a mechanical trigger mechanism comprising a trigger lever pivotally mounted to the ergonomic hand grip; and a swivel hammer coupled to the trigger lever, wherein the swivel hammer is configured to convert trigger lever movement into linear actuation force imparted on the syringe.

### CROSS REFERENCE TO RELATED APPLICATIONS

The present application claims the benefit of U.S. Provisional Patent Application Ser. No. 63/756,619, filed Feb. 10, 2025, the disclosure of which is incorporated herein by reference.

### FIELD

The present disclosure pertains to delivery systems and methods of delivering fluids, and more particularly to delivery systems and methods for controlled formation and delivery of a hydrogel to a body lumen for various medical uses.

### BACKGROUND

Various solutions presently exist for spacing, lifting, and embolic applications. For instance, radiation therapy for prostate cancer involves delivering high doses of radiation to malignant tissue while minimizing exposure to surrounding healthy tissue. Hydrogel spacing materials, such as SpaceOAR™ and SpaceIT™, are injectable polyethylene glycol-based compounds that create temporary space between the prostate and rectum during radiation treatment. These hydrogels are delivered through syringes that allow placement in the target anatomical space.

### SUMMARY

The present disclosure is directed to actuators for controlled formation and delivery of a hydrogel or other fluid to a body lumen for various medical uses.

In an example, a hydrogel deployment actuator may comprise a housing having an ergonomic hand grip; a syringe tray configured to be movably disposed in the housing and configured to removably receive and secure a syringe; a dual-axis swivel head assembly comprising: a dual-axis swivel head; a vertical swivel knob configured to move the dual-axis swivel head along a vertical plane; a horizontal swivel knob configured to move the dual-axis swivel head along a horizontal plane; a swivel lock mechanism configured to lock both vertical and horizontal movement of the dual-axis swivel head; and a flexible tube configured to fluidically couple the dual-axis swivel head to a proximal end of the syringe; and a mechanical trigger mechanism comprising: a trigger lever pivotally mounted to the ergonomic hand grip; and a swivel hammer coupled to the trigger lever, wherein the swivel hammer is configured to convert trigger lever movement into linear actuation force imparted on a plunger of the syringe.

Alternatively or additionally, wherein the vertical swivel knob and the horizontal swivel knob are positioned at a proximal end of the actuator.

Alternatively or additionally, wherein the trigger lever is positioned on the ergonomic hand grip.

Alternatively or additionally, wherein the vertical swivel knob and the horizontal swivel knob are positioned on opposing vertical surfaces of the ergonomic hand grip.

Alternatively or additionally, wherein the swivel lock mechanism is configured to selectively lock and unlock both the horizontal swivel knob and the vertical swivel knob.

Alternatively or additionally, wherein the swivel lock mechanism is a slidable mechanism configured to translate between a locked position and an unlocked position.

Alternatively or additionally, wherein: the swivel lock mechanism is an individual lock configured to simultaneously lock and unlock both the horizontal swivel knob and the vertical swivel knob; and the swivel lock mechanism is located on the ergonomic hand grip at between the horizontal swivel knob and the vertical swivel knob.

Alternatively or additionally, wherein the syringe tray is configured to: contact and maintain a body of the syringe in fixed alignment with the syringe tray; contact a proximal end of a plunger of the syringe; and permit metered longitudinal translation of a plunger of the syringe while maintaining the body of the syringe in the fixed alignment.

Alternatively or additionally, wherein the syringe tray includes a plurality of notches disposed along opposing substantially longitudinally extending surfaces of the syringe tray, and wherein each of the notches is configured to receive at least a portion of the swivel hammer.

Alternatively or additionally, wherein the housing is formed of a casing top rotatably coupled to a casing bottom, and wherein a proximal end of the casing bottom is rotatably coupled to a proximal end of the casing top.

Alternatively or additionally, wherein the housing includes a viewing window.

Alternatively or additionally, wherein the viewing window is located in a top surface of the housing and is aligned with the syringe tray such that at least a body of the syringe is visible through the viewing window when the syringe is loaded in the syringe tray.

Alternatively or additionally, further comprising an ultrasound probe attachment mechanism configured to rigidly mount an ultrasound probe to an exterior surface of the housing.

Alternatively or additionally, wherein the ultrasound probe attachment mechanism further comprises one or more projections configured to receive a flexible strap.

Alternatively or additionally, wherein the ultrasound probe attachment mechanism is located on a bottom surface of the housing.

In another example, a hydrogel deployment actuator may comprise a housing having: an ergonomic hand grip projecting at an angle from a proximal end of the housing; and a cavity in the housing; and an aperture extending through a distal end of the housing; a syringe tray configured to be slidably disposed in the cavity of the housing and configured to removably receive and secure a syringe, wherein the syringe tray includes a plurality of notches disposed along at least a portion of a longitudinal length of the syringe tray; a dual-axis swivel head assembly comprising: a dual-axis swivel head located at a distal end of the housing, wherein a distal end of the dual-axis swivel head includes a luer end extending through the aperture at the distal end of the housing, wherein the proximal end of the luer end is in fluidic communication with a distal end of the syringe and the distal end of the luer end is configured to receive a needle; a vertical swivel knob configured to move the dual-axis swivel head along a vertical plane, wherein the vertical swivel knob is located on the ergonomic hand grip and is coupled via a first drive wire to the dual-axis swivel head; a horizontal swivel knob configured to move the dual-axis swivel head along a horizontal plane, wherein the horizontal swivel knob is located on the ergonomic hand grip and is coupled to the dual-axis swivel head via a second drive wire; a swivel lock mechanism configured to simultaneously lock both vertical and horizontal movement of the dual-axis swivel head; and a flexible tube configured to fluidically couple the dual-axis swivel head to a proximal end of the syringe; a mechanical trigger mechanism comprising: a trigger lever pivotally mounted to the housing; and a swivel hammer coupled to the trigger lever, wherein: the swivel hammer is configured to convert trigger lever movement into metered distal translation of the syringe tray to impart a metered linear actuation force on a plunger of the syringe by the syringe tray, and the swivel hammer is disposable at least partially in a notch of the plurality of notches in the syringe tray.

Alternatively or additionally, further comprising an ultrasound probe attachment mechanism configured to rigidly mount an ultrasound probe to the actuator.

Alternatively or additionally, wherein: the vertical swivel knob and the horizontal swivel knob are positioned on opposing vertical surfaces of the ergonomic hand grip; and the swivel lock mechanism is a slidable mechanism located on the ergonomic hand grip that is configured to translate between: a locked position to simultaneously lock drive wires coupled to both the vertical swivel knob and the horizontal swivel knob; and an unlocked position to simultaneously unlock drive wires coupled to both the vertical swivel knob and the horizontal swivel knob.

In another example a kit for delivering a hydrogel may comprise: a hydrogel deployment actuator comprising: a housing having an ergonomic hand grip; a syringe tray configured to be movably disposed in the housing and configured to removably receive and secure a syringe, wherein the syringe tray includes a plurality of notches disposed along at least a portion of a longitudinal length of the syringe tray; a dual-axis swivel head assembly comprising: a dual-axis swivel head; a vertical swivel knob configured to move the dual-axis swivel head along a vertical plane, wherein the vertical swivel knob is coupled via a first drive wire to the dual-axis swivel head; a horizontal swivel knob configured to move the dual-axis swivel head along a horizontal plane, wherein the horizontal swivel knob is coupled to the dual-axis swivel head via a second drive wire; a swivel lock mechanism configured to simultaneously lock both vertical and horizontal movement of the dual-axis swivel head; and a flexible tube configured to fluidically couple the dual-axis swivel head to a proximal end of the syringe; a mechanical trigger mechanism comprising: a trigger lever pivotally mounted to the housing; and a swivel hammer coupled to the trigger lever, wherein: the swivel hammer is configured to convert trigger lever movement into metered distal translation of the syringe tray to impart a metered linear actuation force on a plunger of the syringe by the syringe tray, and the swivel hammer is disposable in a notch of the plurality of notches in the syringe tray; and an ultrasound probe attachment mechanism configured to rigidly mount an ultrasound probe to the actuator; and a syringe including an injectable hydrogel.

Alternatively or additionally, wherein the syringe is an individual syringe, and wherein the syringe tray is configured to receive the individual syringe.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

FIG. 1 is a partial cross-section view illustrating an example hydrogel composition having been delivered between a rectum and a prostate of a patient in the Denonvilliers'space;

FIG. 2A is a perspective view of the illustrative hydrogel deployment actuator;

FIG. 2B is a view of a distal end portion of the illustrative hydrogel deployment actuator;

FIG. 3A is cutaway view of the illustrative hydrogel deployment actuator with the trigger in a first position;

FIG. 3B is another cutaway view of the illustrative hydrogel deployment actuator with the trigger in a second position;

FIG. 4A a view of the illustrative hydrogel deployment actuator with the housing in an open position and a syringe disposed in the syringe tray; and

FIG. 4B is a view of the illustrative hydrogel deployment actuator with the housing in a closed position.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

The present disclosure is directed to systems and methods for delivering a conformable, fillable balloon suitable for implantation in a mammalian body.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings. The drawings, which are not necessarily to scale, depict illustrative embodiments and are not intended to limit the scope of the disclosure.

Prostate cancer is the most common cancer and the second leading cause of cancer death among men in the United States. While radiation therapy serves as a primary treatment approach, it presents significant technical challenges in delivering targeted radiation doses while protecting surrounding healthy tissue. A critical limitation in radiation therapy is the inevitable exposure of surrounding tissues to radiation during treatment. To address this, hydrogel spacing materials are deployed between the prostate and rectum.

Various solutions presently exist for spacing, lifting, and embolic applications. However, injectable materials, including in-situ crosslinking materials and shear thinning materials, can have some potential inconveniences that include asymmetric localized deployment of the implanted material, potential for off-target embolization due to delayed reaction or migration of the implanted material, and complexity associated with removal of material if complete removal of the material is desired. In one illustrative example, injectable materials may be used between the rectum and the prostate to prevent damage during radiation therapy. One illustrative system for injecting materials into the space between the rectum and the prostate is the SpaceOAR Vue™ System available from Boston Scientific Corporation, Marlborough, MA, USA. In the SpaceOAR Vue™ System, the product may be in a liquid form before it sets into a solid/gel implant. The flowable pre-gel may be somewhat restricted in the superior direction (e.g., by the seminal vesicles and associated structures) and in the inferior direction (e.g., by the Denonvilliers' fascia).

However, current hydrogel deployment systems face several significant technical challenges that limit their effectiveness. The deployment of existing hydrogel systems is particularly challenging at least due to three key mechanical limitations: 1) the high force required to overcome hydrogel viscosity during manual injection, 2) the precise anatomical positioning needed in close proximity to sensitive structures, and 3) the lack of proper mechanical support for the injection syringe. Some current electronic control systems attempt to address these challenges through complex force and velocity monitoring. However, these electronic approaches introduce additional complications such as reliance on power systems and electronic components that can fail, complex feedback mechanisms requiring calibration, and/or an inability to provide direct tactile feedback to the physician. Furthermore, during hydrodissection procedures, precise needle manipulation is essential but extremely difficult with existing systems. Moreover, in some procedures the close proximity of a transrectal ultrasound (TRUS) probe to the perineum, combined with the need for sharp needle positioning, makes conventional syringe maneuvering both challenging and potentially dangerous. Existing electronic control systems focus primarily on maintaining predetermined force and velocity parameters but fail to address the fundamental need for precise mechanical control during needle positioning and hydrogel delivery. This highlights the need for a purely mechanical solution that provides both controlled deployment force and precise needle maneuverability while maintaining rigid anatomical positioning

The present disclosure provides a purely mechanical hydrogel deployment system that overcomes multiple technical challenges in existing systems through an innovative combination of mechanical components. In one aspect, the disclosure provides a dual-axis swivel head assembly that permits precise dual-axis needle positioning through direct mechanical control, eliminating the need for complex electronic systems. The swivel head assembly includes vertical and horizontal adjustment knobs that provide dual-axis positional adjustment of a needle while maintaining rigid mounting to an ultrasound probe, thereby addressing the critical challenge of needle maneuverability in confined anatomical spaces. In another aspect, the disclosure incorporates a novel bottom-loading syringe tray mechanism that permits secure syringe mounting and exchange while maintaining probe position. This mechanical solution eliminates the problem of unsupported syringes that plague existing systems. The disclosure further provides a mechanical trigger mechanism incorporating a swivel hammer design that converts pivotal movement into linear actuation force. This purely mechanical system provides the necessary mechanical advantage to overcome hydrogel viscosity while enabling metered deployment, eliminating the need for complex electronic force monitoring systems. In another aspect, a universal (e.g., strap) attachment mechanism permits rigid mounting to different ultrasound probes while maintaining stability during needle manipulation. The mechanical locking system allows the needle orientation to be fixed in both vertical and horizontal directions once the desired position is achieved. The above-mentioned mechanical elements provide several key advantages over existing electronic systems including direct tactile feedback during needle positioning and hydrogel deployment, reduced procedural complexity through elimination of electronic components, improved reliability through purely mechanical operation, metered injection of hydrogel, and/or enhanced control through infinite positional adjustment capability. These mechanical solutions work together to provide precise anatomical access and controlled hydrogel delivery while maintaining the stability and ease of use required for clinical applications.

While the present disclosure is described with respect to injecting materials between the rectum and the prostate to prevent damage during radiation therapy, the systems and methods described herein may be used in other anatomies, as desired. The systems and methods described herein may be useful for a number of medical procedures including spacing, lifting, bulking, embolization and backfill procedures. Spacing procedures include soft tissue spacing procedures wherein one or more fillable balloons are placed, for example, between the rectum and the prostate and filled to prevent damage during radiation therapy or between other organs and/or tissues to protect them from potential side effects of a particular treatment, and joint spacing procedures where one or more fillable balloons are placed in the space of a joint, such as a shoulder, hip, knee or ankle joint and filled to provide mechanical support for the joint. Lifting procedures include procedures where one or more fillable balloons are placed and filled to lift a sinus membrane for sinus augmentation or others. Bulking procedures include procedures wherein one or more fillable balloons are implanted in or adjacent to a bodily sphincter, such as an anal sphincter or a urethral sphincter, to address intrinsic sphincter deficiency or others. Embolization procedures include those where one or more fillable balloons are placed in a target blood vessel and filled to block blood flow to an area of the body. Backfill procedures include procedures wherein one or more fillable balloons are implanted and filled in a left atrial appendage after the introduction of a closure device such as the Watchman® left atrial appendage closure device available from Boston Scientific Corporation, Marlborough, MA, USA or the balloon is used as a closure device for the left atrial appendage with no additional support or devices.

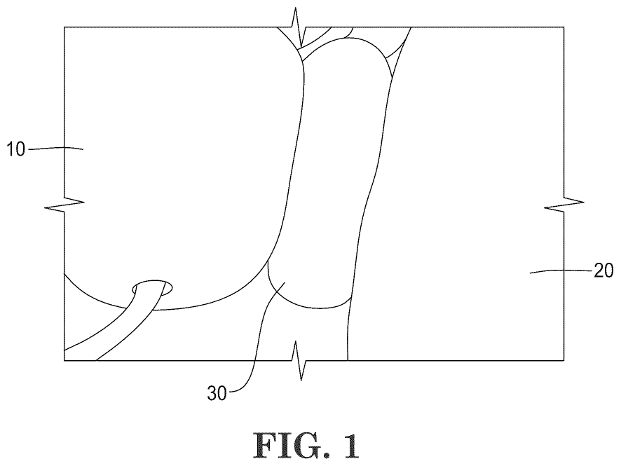

Turning to the drawings, FIG. 1 is a partial cross-section view illustrating an example of hydrogel 30, having been delivered between a rectum 20 and a prostate 10 of a patient in the Denonvilliers' space. The hydrogel 30 may be an injectable hydrogel that is injected from a syringe such as those described herein.

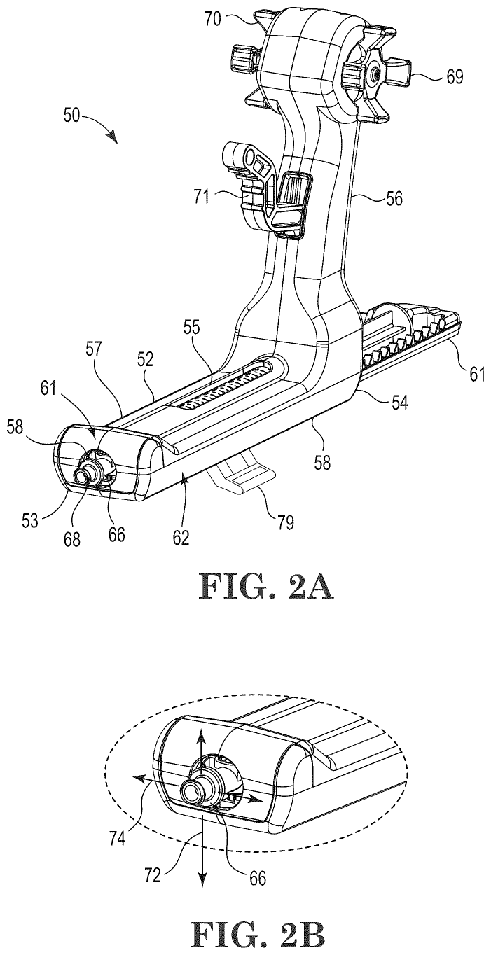

FIG. 2 is a perspective view of an illustrative hydrogel deployment actuator 50. As illustrated in FIG. 2A, the actuator 50 includes a housing 52. The housing 52 has a distal end 53 and a proximal end 54. A longitudinal length of the housing 52 extends between the distal end 53 and the proximal end 54. The housing 52 can have a plurality of surfaces including a first (top) surface 57 and a second (bottom) surface that opposes the first surface 57.

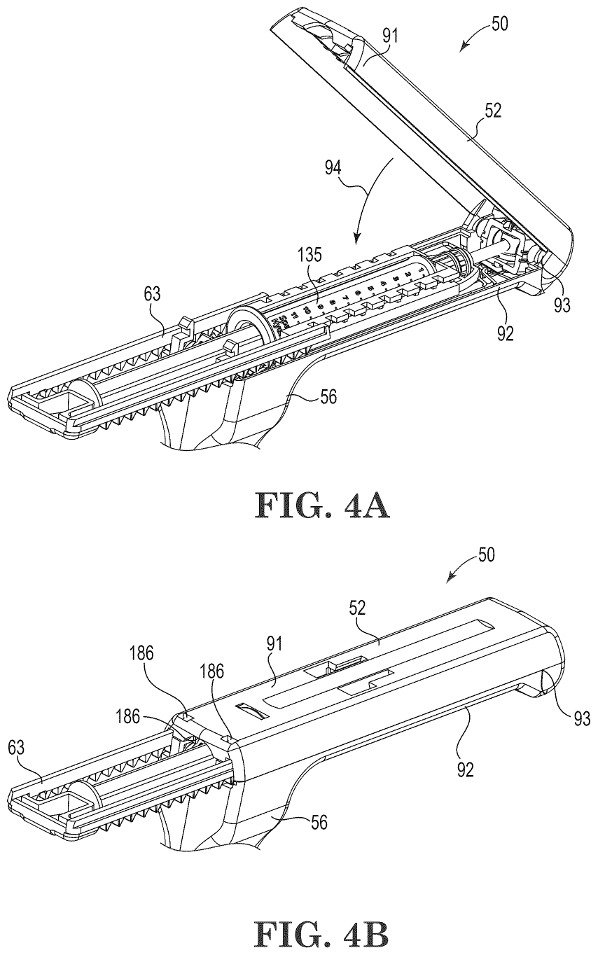

In some embodiments, the housing 52 is formed of two or more components that are coupled together. For instance, the housing 52 can be formed of a casing top 91 and a casing bottom 92 that are coupled together. The casing top 91 and the casing bottom 92 can be pivotably coupled together such that the housing 52 is configured to selectively open and close to permit a syringe to be inserted in a cavity within the housing 52. For example, the casing top 91 and the casing bottom 92 can be pivotably connected about a pin, shaft, or other type of pivot point located proximate to the distal end 53 of the housing 52. Hence, the distal portion or distal end of the casing bottom 92 can be rotated about the pivot point 93 in a first direction (e.g., as represented by element 94) such that proximal end of the casing bottom 92 is spaced a distance away from the casing top 91 when the housing 52 is in an open configuration, for instance as illustrated and described in greater detail in FIG. 4A. Thus, a syringe can readily be inserted in or removed from a cavity in the housing 52 when the housing 52 is configured in the in open configuration. Once the syringe is inserted or removed from the housing 52, the distal portion or distal end of the casing bottom 92 can be rotated about the pivot point 93 in a second direction (opposing the first direction) such that proximal end of the casing bottom 92 is in contact with the casing top 91 when the housing 52 is in a closed configuration, for instance as illustrated and described in greater detail in FIG. 4B.

The housing 52 can include a viewing window 55. The viewing window 55 can be an elongated viewing window 55 extending in a substantially longitudinal direction, as illustrated in FIG. 2A. Hence, the viewing window can extend along a portion of a longitudinal length of the housing 52. The viewing window 55 can be formed of a translucent material or other material that permits at least the body portion of the syringe to be visible to an operator of the actuator 55 e.g., when the housing 52 is in a closed configuration. The viewing window 55 can be formed in a top surface of the casing top 91 that forms the upper portion of the housing 52, as illustrated in FIG. 2A. For instance, the viewing window 55 can be located on a side of the actuator 55 the is opposite the casing bottom of the housing 52 and/or that is opposite an ultrasound attachment mechanism 79. Hence, the viewing window 55 can permit visibility of at least the body of the syringe that is disposed in the actuator 50 even when the actuator 50 is coupled to an ultrasound probe (not illustrated in FIG. 2A). The viewing window 55 can be aligned with the syringe tray 61 such that at least the body of the syringe is visible when the syringe is loaded in the syringe tray and when the housing 52 is configured in the closed configuration.

The housing 52 can include an ultrasound probe attachment mechanism 79. The ultrasound probe attachment mechanism 79 can be coupled to or integral with the casing bottom of the housing 52, as illustrated in FIG. 2A. The ultrasound probe attachment mechanism 79 can be configured to rigidly mount an ultrasound probe to the housing 52. The ultrasound probe attachment mechanism 79 can be manifested as flexible loop or ring or as a strap or other configuration of material. For instance, the ultrasound probe attachment mechanism 79 can include one or more projections that project from the housing (e.g., substantially radially project from the housing 52) and include one or more orifices (e.g., elongated slots) configured to receive a flexible strap (not illustrated in FIG. 2A). For example, the ultrasound probe attachment mechanism 79 can include two projections which are circumferentially spaced apart and each having openings therein that configured to receive respective portions of the flexible strap. The strap can be secured to the housing 52 via the projections and can be sized or otherwise configured (e.g., with a hook and loop fasteners, or other type of fastener) to receive and rigidly mount ultrasound probes of various dimensions to the housing 52. For example, the flexible strap can be looped through the ultrasound probe attachment mechanism 79, an ultrasound probe can be positioned within or adjacent to the flexible strap, and the flexible strap can be tightened and secured (e.g., with hook and loop fasteners, etc.) to rigidly mount the ultrasound probe to the bottom surface of the housing 52. That is, once an ultrasound probe is coupled to the housing 52 via the comprising an ultrasound probe attachment mechanism 79, the ultrasound probe can be fixed to the particular location (e.g., does not move relative to the housing 52). For example, the ultrasound probe can remain rigidly mounted to the housing 52 prior to, during, and/or subsequently to moving the housing 52 between an open and closed configuration. Thus, the housing 52 can be configured to permit access to and change a syringe located within the housing 52, even when an ultrasound probe is coupled to the housing 52, yet, alignment between the actuator 50 and the ultrasound probe can be maintained e.g., during vertical and horizontal needle movements, trigger actuation for hydrogel deployment, manipulation of control knobs. The ultrasound mounting system herein desirably yields rigid holding with different transrectal TRUS probes, for instance, due to the flexible strap design, stable positioning during knob-controlled needle maneuvering with or without moving the TRUS probe, and permits deployment of hydrogel at different positions in vivo while maintaining anatomical access (e.g., the secure attachment mechanism that prevents unwanted movement during needle manipulation and hydrogel deployment). That is, the mounting system herein permits precise needle control while maintaining a stable fixed probe position, allowing the physician to focus on proper hydrogel placement without concerns about device stability or alignment.

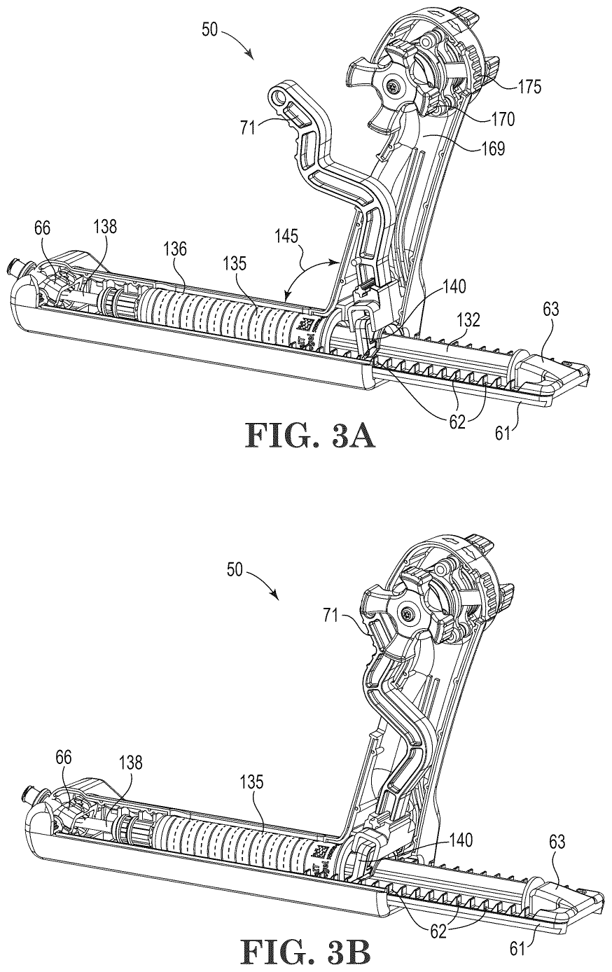

As illustrated in FIG. 2A, an ergonomic hand grip 56 can project (e.g., substantially radially project) from the housing 52. For instance, the ergonomic hand grip 56 can project at an angle from a proximal end of the housing 52. The angle can be a non-zero angle. For instance, the angle (e.g., the angle 145 as illustrated in FIG. 3A) can be an obtuse angle or an acute angle. For instance, the angle can be about 90 degrees, about 100 degrees, about 110 degrees, or about 120 degrees, among other possibilities. The ergonomic hand grip 56 can project from a first (top) surface 57 of the casing top 91 of the housing 52. For instance, the ergonomic hand grip 56 can be integral with the casing top 91 or can be coupled to the casing top 91.

As illustrated in FIG. 2A, the housing 52 can include an aperture 58 extending through the distal end 53 of the housing 52. The aperture 58 can be sized to permit at least a portion of a dual-axis swivel head to extend through the aperture 58. For instance, the aperture 58 can be a circular aperture with a diameter that is larger than a distal end of the dual-axis swivel head 66. Stated differently, the aperture 58 can be configured to permit the dual-axis swivel head 66 to move along both a vertical axis and a horizontal axis.

For instance, FIG. 2B is a view of the distal end 53 of the illustrative hydrogel deployment actuator 50. As illustrated in FIG. 2B, the aperture 58 can be a circular aperture located centrally in the distal end 53 of the actuator 50. That is, the aperture 58 can be located along a longitudinal axis of the actuator 50, for instance to align the center of the aperture with a center of the dual-axis swivel head 66 (e.g., when the dual-axis swivel head 66 is in a neutral configuration e.g., has zero degree offset both vertically and horizontally from the longitudinal axis of the actuator 50).

As illustrated in FIGS. 2A-2B, the distal end of the dual axis swivel head 66 can include or be formed of a luer end 68 that extend through the aperture at the distal end 53 of the housing 52. The proximal end of the luer end 68 can be in fluidic communication with a distal end of the syringe. The distal end of the luer end 68 can be configured to receive a needle or other fluid injection member. Hence, fluid (e.g., a hydrogel) can be transferred from the syringe via at least the luer end 68 to a needle thereby permitting injection of the fluid e.g., in a metered fashion as described herein. Stated differently, the syringe disposed in the actuator 50 can be in fluid communication via various components of the actuator 50 with a needle coupled to the distal end of the luer end 68, as detailed herein.

The dual-axis swivel head 66 can be disposed at least partially within the aperture 58. For instance, a distal end of the dual-axis swivel head 66 can extend distally outside of the aperture 58, as illustrated in FIGS. 2A-2B. That is, the dual-axis swivel head 66 can be located at the distal end 53 of the housing 52 and a distal end of the dual-axis swivel head 66 can extend outside of the aperture 58. The dual-axis swivel head 66 can be configured with at least two degrees of freedom e.g., relative to the distal end 53 of the housing 52. For instance, the dual-axis swivel head 66 can be configured to move (rotate) along a vertical plane (represented by element 72 in FIG. 2B) and can be configured to move (rotate) along a horizontal plane (represented by element 74 in FIG. 2B). For instance, the dual-axis swivel head 66 can be pivotably coupled to the housing 52 to permit at least the distal end or luer end 68 thereof or the entire dual-axis swivel head 66 to rotate relative to the housing 52. Hence, the dual-axis swivel head assembly 66 can permit precise needle positioning through direct mechanical control of the dual-axis swivel head assembly 66, as detailed herein.

For instance, the horizontal and vertical position of the dual-axis swivel head 66 can be controlled via corresponding vertical and horizontal swivel knobs. For example, a vertical swivel knob 70 can be configured to move the dual-axis swivel head 66 along the vertical plane 72 and a horizontal swivel knob 69 configured to move the dual-axis swivel head 66 along a horizontal plane 74. The vertical swivel knob 70 and the horizontal swivel knob 69 can be positioned at the proximal end 54 of the actuator 50. For instance, the vertical and horizontal swivel knobs 70, 69 can be located on the handle or ergonomic hand grip 56. The vertical swivel knob 70 and the horizontal swivel knob 69 can be positioned on opposing vertical surfaces of the ergonomic hand grip 56, as illustrated in FIG. 2A. Having the vertical swivel knob 70 and the horizontal swivel knob 69 be positioned on opposing vertical surfaces of the ergonomic hand grip 56 can promote aspects herein, such as promoting independent control of the knobs 70, 69 and/or permitting the knobs 70, 69 to be locked/unlocked at the same time, as detailed herein. While the knobs 70, 69 are illustrated as distinct knobs located at different positions, in some embodiments the functionality of the knobs 70, 69 can be combined into an individual knob configured to adjust the alignment of the dual-axis swivel head 66 along both the vertical axis and the horizontal axis independently or simultaneously.

The vertical and horizontal swivel knobs 70, 69 can mechanically connect to and control needle positioning. Each swivel knob is connected to the dual-axis swivel head 66 via dedicated drive wires (e.g., drive wires 170, 169, as illustrated in FIGS. 3A-3B). A first driver wire can couple a first knob to the dual-axis swivel head and a second drive wire can couple the second knob to the dual axis swivel head. For instance, a first drive wire 170 connects the vertical swivel knob 70 to control up/down movement of the dual-axis swivel head 66, while a separate second drive wire 169 connects the horizontal swivel knob 69 to control side-to-side movement dual-axis swivel head 66. When the vertical or horizontal knob is rotated, it applies tension to its respective drive wire, which mechanically translates the rotational motion of the knob into positional adjustment of the dual-axis swivel head 66 along the corresponding vertical or horizontal axis. The drive wires provide purely mechanical force transmission between the control knobs 70, 69 and dual-axis swivel head 66, allowing for tactile feedback and precise control without requiring electronic components.

As detailed herein, the actuator 50 can include a flexible tube (e.g., the flexible tube 138, as illustrated in FIGS. 3A-3B). The flexible tube can be configured to fluidically couple the dual-axis swivel head 66 to a proximal end of the syringe. For instance, the flexible tube can be formed of plastic or another type of semi-rigid material can permit the tube (e.g., at least a distal end portion of the tube) to move responsive to movement of the dual-axis swivel head 66. Hence, the flexible tube connecting the syringe to the swivel head and the luer end maintains fluid communication while accommodating this dual-axis movement of the dual-axis swivel head 66. The flexible tube facilitates the multi-directional needle movement (e.g., of the dual-axis swivel head) while maintaining fluid communication with a syringe.

For instance, the flexible tube is positioned between the syringe and the swivel head and luer end (needle) to accommodate both vertical and horizontal swivel movements controlled by the respective knobs. When the vertical or horizontal swivel knobs are actuated via their drive wires, the flexible tube can bend or flexes to allow the luer end to move along that axis while maintaining an uninterrupted fluid path. Hence, the flexible tube's design permits positional adjustment of the needle through the dual-axis swivel head movement while ensuring consistent fluid (e.g., hydrogel) flow from the syringe to the luer end. The flexible tube's flexibility allows coordinated movement between the swivel head and luer end during needle manipulation while preserving the sealed fluid communication pathway needed for controlled hydrogel deployment. This mechanical solution permits precise needle positioning through knob-controlled infinite adjustment while rigidly mounted on the TRUS probe, addressing the critical challenge of needle maneuverability in confined anatomical spaces without compromising fluid delivery.

A swivel lock mechanism (e.g., the swivel lock mechanism 175, as illustrated in FIGS. 3A-3B) can be configured to simultaneously lock both vertical and horizontal movement of the dual-axis swivel head 66. For instance, the swivel lock mechanism 175 can simultaneously lock both vertical and horizontal movement by securing the drive wires in position once the desired needle orientation is achieved. Thus, when configured in a locked position the swivel lock mechanism 175 can simultaneously lock both the vertical swivel knob 70 and the horizontal swivel knob 69 through a single locking action (e.g., by translation of the swivel locking mechanism 175 in a first direction). When engaged, the swivel lock mechanism 175 can be configured to fix the position of both drive wires simultaneously to maintain the set needle orientation, permit stable needle positioning during trigger actuation and hydrogel delivery and preserves the precise anatomical access achieved through knob-controlled dual-axis adjustment. Conversely, when configured in an unlocked position the swivel lock mechanism 175 can simultaneously unlock both the vertical swivel knob 70 and the horizontal swivel knob 69 through a single locking action (e.g., by translation of the swivel locking mechanism 175 in a second direction opposing the first direction). This mechanical locking system allows a physician or other individual to set and maintain exact needle positioning for optimal hydrogel placement, lock both axes of movement with a single mechanism, and deploy hydrogel while keeping the needle securely oriented in the desired position. In some embodiments, the swivel lock mechanism 175 can be manifested as a slidable mechanism that is configured to translate between a locked position and an unlocked position. However, types of mechanisms and methods of actuation of the swivel lock mechanism 175 are possible. For instance, the swivel lock mechanism 175 can be a rotatable swivel lock mechanism that is configured to rotate to selectively lock or unlock the knobs 70, 69. In any case, when in the locked position, the swivel lock mechanism can contact the drive wires to tension or lock the drives wires in a given position, whereas when in an unlocked position the swivel lock mechanism can be located a distance away from the drive wires and thus permit movement of the drive wires. In some embodiments, the swivel lock mechanism 175 can be located on the ergonomic hand grip 56. For instance, the swivel lock mechanism 175 can be located on the handle 56 between (e.g., equally spaced between) the knobs 70,69 thereby permitting one finger or one hand locking and unlocking of the swivel lock mechanism 175, for instance, while also maintaining one or both of the knobs 70, 69 in a desired position. However, other positions of the swivel lock mechanism 175 are possible. Additionally, while the swivel lock mechanism 175 is illustrated as an individual lock, in some embodiments the swivel lock mechanism can be manifested as two locks which correspond to and can independently lock and unlock the knobs 70, 69.

The actuator 50 includes a mechanical trigger mechanism. The mechanical trigger mechanism comprises a trigger lever 71 pivotally mounted to the ergonomic hand grip 56 and a swivel hammer (e.g., the swivel hammer 140, as illustrated in FIGS. 3A-3B) coupled to the trigger lever 71. As detailed herein, the swivel hammer is configured to convert movement of the trigger lever 71 into linear actuation force imparted on a plunger of the syringe, as detailed herein. As illustrated in FIG. 2A, the trigger lever 71, the vertical swivel knob 70, and the horizontal swivel knob 69 can each be positioned on the ergonomic hand grip or handle 56. In some embodiments, each of the trigger lever 71, the vertical swivel knob 70, the horizontal swivel knob 69, and the swivel lock mechanism 175 can be positioned on the ergonomic hand grip or handle 56. The trigger lever 71 can be configured to move (translate in a substantially longitudinal direction) responsive to actuation by an operator of the actuator 50. In some embodiments, the trigger lever 71 can include a spring other mechanism to disposition the trigger lever 71 to an unactuated position (e.g., as shown in FIG. 2A and FIG. 3A). Responsive to actuation by the operator, the trigger lever 71 can be moved (e.g., substantially proximally) to an actuated position (e.g., as shown in FIG. 3B). As detailed herein with respect to FIGS. 3A-3B, the actuation of the trigger lever 71 can impart movement in the swivel hammer which in turn cases the syringe tray 61 to move longitudinally (e.g., distally) to impart a force on the plunger of the syringe disposed in the actuator 50. The interrelation between and/or the configuration of the trigger lever 71, the swivel hammer, and the syringe tray 61 can provide a mechanical advantage and also permit metered delivery of a fluid (e.g., hydrogel), as detailed herein. For instance, the swivel hammer is configured to be disposable in a notch of the plurality of notches in the syringe tray 61

The actuator 50 includes a syringe tray 61. The syringe tray 61 can be configured to receive a syringe. In some embodiments, the syringe tray 61 can be configured to receive an individual syringe (e.g., the individual syringe 135, as illustrated in FIGS. 3A-3B). The syringe tray 61 can be an elongated syringe tray that is configured to maintain a body of the syringe in fixed alignment with the housing 52 and permit metered longitudinal translation of a plunger of the syringe while maintaining the body of the syringe in the fixed alignment with the housing 52. For instance, the syringe tray 61 can be configured to hold the body of the syringe in a fixed position (e.g., relative to the housing 52 and/or the syringe tray 61) and can permit longitudinal translation of the plunger of the syringe (e.g., relative to the housing 52 and/or the syringe tray 61). The syringe tray 61 can be a bottom-loading syringe tray which is configured to permit loading or unloading of a syringe from the bottom of the actuator 50. Hence, the syringe tray 61 can be configured to permit easy attachment of a syringe (e.g., SpaceIT™ containing syringe) while maintaining proper alignment of the syringe tray 61 with the swivel hammer, securely hold the syringe in fixed position during deployment to ensure consistent interface with the swivel hammer, and/or allow syringe exchange while the actuator remains mounted to the ultrasound probe.

The syringe tray 61 can be configured to move longitudinally within the housing 52. For instance, the syringe tray 61 can be disposed within a cavity of the housing 52 and can be configured to move longitudinally within the cavity responsive to actuation of the trigger lever 71, as detailed herein. The syringe tray 61 can include a plurality of notches 62 or other projections. The notches 62 can be disposed along at least a portion of the longitudinal length of the syringe tray 61. For instance, the notches 62 can be manifested as a series of longitudinally extending notches that are the same size, same shape, and are uniformly spaced along the length of the syringe tray 61. For example, the notches 62 can be formed of two sets of notches on opposing inner or interior longitudinally extending surfaces of the syringe tray 61. The notches 62 can each project inward (radially), as illustrated in FIGS. 3A-3B. The notches 62 can have a beveled or slated distal surface and a flat or planar proximal surface, as illustrated in FIGS. 3A-3B. Hence, the notches 62 can be configured to promote movement of the swivel hammer relative to the notches 62, as detailed herein with respect to FIGS. 3A-3B.

In some embodiments, the syringe tray 61 can include a projection at a proximal end of the syringe tray 61. The projection 63 can be configured to contact the proximal end of the plunger of the syringe when the syringe is disposed in the syringe tray 61. For instance, the projection 63 can be a planar projection configured to contact a planar proximal end of the plunger. The projection 63 can be the same shape and size and the proximal end of the plunger e.g., as illustrated in various figures herein or can be a different shape and size (e.g., smaller diameter) than the proximal end of the plunger. The projection 63 can be located proximal to the proximal end of the plunger of the syringe and can be in contact with the proximal end of the plunger of the syringe (e.g., as illustrated in FIGS. 3A-3B). Hence, longitudinal movement of the syringe tray 61 can impart longitudinal movement (e.g., in the same direction and magnitude) of the plunger of the syringe.

The actuator 50 can include a swivel hammer 140. The swivel hammer 140 can be affixed or coupled to an end of the trigger lever 71. For instance, the swivel hammer 140 can be pivotably coupled to the end of the trigger lever 71 that is most proximate to the syringe tray 61. A portion of the swivel hammer 140 can project into a notch of the notches 62 of the syringe tray 61. That is, the swivel hammer 140 can be configured (e.g., is sized and shaped) such that at least a portion of the swivel hammer projects into the notches 62. In some embodiments, the swivel hammer can be configured as a substantially rectangular or substantially square member. The swivel hammer 140 can be configured to maintain consistent force transmission through direct mechanical contact with the syringe plunger. The interface between the syringe and swivel hammer is maintained through proper geometric alignment between the syringe tray and swivel hammer, direct mechanical contact between the swivel hammer and syringe tray 61 holding the syringe plunger when loaded. This configuration desirably yields controlled force transmission from the trigger lever 71 to the syringe plunger, metered deployment of hydrogel through mechanical advantage and, as detailed herein, permits visual monitoring of hydrogel levels through the viewing window while maintaining secure syringe positioning.

The above-mentioned mechanical system embodied in the actuator 50 desirably permits positional adjustment of a needle through direct manual control of the knobs 70, 69, independent control of vertical and horizontal needle movement, precise needle positioning while maintaining rigid mounting to the ultrasound probe, and stable needle orientation when locked (e.g., by the locking mechanism described herein). For instance, the actuator 50 can be configured to permit controlled needle positioning, via the dual-axis swivel head assembly, while maintaining anatomical access and metered hydrogel deployment, responsive to the trigger lever movement, while maintaining anatomical access. That is, the actuator 50 can be configured to permit, via the dual-axis swivel head 66, dual-axis positional adjustment of an attached needle coupled to the dual-axis swivel head while maintaining rigid mounting to an ultrasound probe.

In addition to the distal aperture 58, the housing 52 can include a plurality of apertures (e.g., circular apertures 186, as illustrated in FIG. 4B) extending through the proximal end 54 of the housing 52, as detailed herein. For instance, the plurality of apertures can include an aperture configured to permit a proximal end or proximal portion (e.g., a plunger) to extend through the aperture and an aperture (e.g., two apertures) configured to permit a proximal end or proximal portion of a tray to extend therethrough. Thus, the proximal portions of the syringe and the tray can extend in a substantially longitudinal manner a distance from the proximal end 54 of the housing 52 e.g., when the housing is in a closed configuration.

FIG. 3A is cutaway view of the illustrative hydrogel deployment actuator 50 with the trigger lever 71 and swivel hammer 140 in a first position, while FIG. 3B is cutaway view of the illustrative hydrogel deployment actuator 50 with the trigger lever 71 and swivel hammer 140 in a first position. The actuator 50 incorporates an innovative mechanical trigger-hammer-notch system that provides precise control over hydrogel delivery without relying on electronic components. As mentioned, the trigger mechanism comprises the trigger lever 71 pivotally mounted to the handle and that is positioned for ergonomic actuation while maintaining stable device control. When squeezed or actuated, the trigger lever 71 pivots around its mounting point to engage or apply a force (e.g., an increased longitudinal force) to the swivel hammer 140. The swivel hammer 140 is uniquely configured to interface with a series of notches 62 disposed along the longitudinal length of the syringe tray 61. These notches 62 serve to provide predetermined engagement points for the swivel hammer 140, permit metered distal translation of the syringe tray 61 during trigger lever 71 actuation, and allow controlled force transmission through indirect mechanical coupling between at least the trigger lever 71 and the plunger of a syringe 135 disposed in the syringe tray 61. As mentioned, the device can include a flexible tube 138 that is coupled to the dual-axis swivel head

For instance, the mechanical interaction between these components in the actuator 50 can follow the following sequence. The trigger lever's 71 pivotal movement (e.g., from the first position as illustrated in FIG. 3A to the second position as illustrated in FIG. 3B) can actuate the swivel hammer 140 such that the swivel hammer engages with (e.g., is move proximally and/or imparts a force substantially proximally on) the notches (e.g., two respective notches positioned at the same longitudinal position along opposing surfaces) of the syringe tray 61. That is the syringe tray 61 can be moved distally relative to the housing 52 and/or the body 136 of the syringe to impart a corresponding distal movement of the plunger 137 of the syringe which injects a metered or predetermined amount of fluid from the body or barrel of the syringe 135. The metered or predetermined amount of fluid (e.g., hydrogel) can be equal to a volume of the syringe that is displaced by the metered movement of the plunger 137. That is, the notched interface between the syringe tray 61 and the swivel hammer 140 converts the pivotal trigger motion into controlled linear movement which also servers to provide a mechanical advantage reduces user effort while maintaining precise control of injection of a fluid from the syringe 135). This purely mechanical system provides several advantages over electronic control systems including direct tactile feedback during fluid (e.g., hydrogel) delivery, reduced procedural complexity through elimination of electronic components, enhanced reliability through purely mechanical operation, precise control over deployment volumes through the notched interface, and stable positioning during actuation while maintaining ultrasound probe alignment. As illustrated in FIGS. 3A-3B, the swivel hammer 140 can be located between the body of the syringe 135 and the plunger of the syringe 135 when the syringe is disposed in the syringe tray 62.

FIG. 4A is a view of the illustrative hydrogel deployment actuator 50 with the housing 52 configured in an open position with a syringe 135 disposed therein, while FIG. 4B is a view of the illustrative hydrogel deployment actuator 50 with the housing 52 configured in a closed position. Namely, the syringe 135 can be inserted within a cradle or syringe lounger disposed in a syringe tray 61 that is disposed in the cavity in the housing 52, as illustrated in FIG. 4A. For instance, the housing 52 can be formed of a casing top 91 and a casing bottom 92 that are coupled together. The casing top 91 and the casing bottom 92 can be pivotably or rotatably coupled together such that the housing 52 is configured to selectively open and close to permit the syringe 135 to be inserted in a cavity within the housing 52, as detailed herein. While FIGS. 4A-4B illustrate the casing top 91 has been rotatably coupled to the casing bottom 92, in some embodiments the casing top 91 and the casing bottom 92 can be movably coupled together via a different mechanism. For instance, in some embodiments, the casing top 91 and the casing bottom 92 can be slidably coupled (e.g., via corresponding channels and/or notches/grooves, etc.) or can be configured to rotate relative to each other in a different direction. For example, the casing top 91 can be slidable coupled to the casing bottom 92 to permit the top casing 91 and the casing bottom 92 to translate in a substantially or entirely longitudinal direction relative to each other between an open position (e.g., where the syringe tray 61 in the housing 62 is accessible) and a closed position (e.g., where the syringe tray 61 in the housing 62 is inaccessible or otherwise overlaid by the housing 62).

In some embodiments, a method of injecting a hydrogel in a subject with the illustrative hydrogel deployment actuator is provided. While certain steps described herein occur in a given sequence, in other embodiments fewer steps are contemplated and the order by which steps are performed can be different than what is described. The method is performed with the hydrogel deployment actuators described herein. The method can includes accessing a target site in vivo. For instance, a needle may be inserted to the target site or target location. The target location may be between the prostate and the rectum. The needle may be an 18-gauge hypodermic needle. However, the needle may be smaller than 18 gauge or greater than 18-gauge, as desired. Exemplary materials for the needle include, but are not limited to, metals and metal alloys, such as stainless steel and Nitinol, and polymers. The distal tip of the needle may be sharpened and may have a beveled shape. Next, a dilator may be advanced over the needle to widen the access path. For example, the dilator may include a lumen extending from a proximal end (not shown) to a distal end of the dilator. The lumen may be sized and shaped to receive the needle therein. An access sheath may be advanced over the dilator. In some cases, the sheath may be advanced substantially simultaneously with the dilator. In other cases, the access sheath may be advanced over the dilator after the dilator has been positioned. The sheath may include a lumen extending from a proximal end (not shown) to a distal end of the sheath. The lumen may be sized and shaped to receive the dilator and the actuator 50 therein. In some examples, the lumen may have diameter of approximately 10 millimeters (mm). However, the diameter of the lumen may be less than 10 mm or greater than 10 mm.

The dilator may then be removed while the sheath is left in place. Next a needle coupled to the distal end of the actuator may be advanced through the lumen of the sheath. The actuator 50 may be distally advanced until the distal tip of the needle is positioned at the target location. Once positioned at the target location, the trigger lever of the actuator 50 can be selectively actuated to inject metered amounts of fluid (e.g., hydrogel) at the target location. In some embodiments, the fluid injected in vivo by the actuator 50 of the present disclosure can be imaged during or after administration of the fluid using a suitable imaging technique such as ultrasound or an X-ray-based imaging technique, such as computerized tomography or X-ray fluoroscopy.

In some embodiments, one or more components of the actuator 50 can include a luer adapter or other component at a proximal end thereof. The luer adaptor can permit introduction of a fluid such as saline and/or a hydrogel. For example, the luer adaptor can be permit the introduction of saline or another fluid for hydro dissection of tissue, in some embodiments.

In some embodiments, one or more components of the actuators herein can include markings such as marker bands disposed on a portion of a surface (e.g., exterior surface) thereof. The presence of the markings such as marker bands can aid in delivery of the components such as a needle and/or a fluid (e.g., a hydrogel), to a target site or location in vivo. For instance, in some embodiments a distal tip or needle of dilator can include markings configured in an echogenic pattern or other pattern (e.g., longitudinally) along a portion of a length of the distal tip or need of the dilator.

As used herein, a “hydrogel” is a crosslinked polymer that contains water or can absorb water but does not dissolve when placed in water, although hydrogels may degrade in vivo over time or be degraded by introduction of ex vivo substances.

In some embodiments the fluid injected by the actuator 50 herein may be in the form of aqueous liquids including aqueous solutions and aqueous dispersions, particular examples of which include normal saline, phosphate buffered saline, 5% dextrose in water (D5W), contrast media including radiographic contrast media such as iodinated contrast media (e.g., Lipiodol® Guerbet LLC, Princeton, NJ, USA), iohexol (Omnipaque™, GE HealthCare Technologies, Inc., Chicago, IL, USA), iodixanol (Visipaque™, GE HealthCare Technologies, Inc.), iopamidol, ioversol, etc.) and barium sulfate, magnetic resonance imaging (MRI) contrast media including gadolinium-containing contrast media, near-infrared (NIR) contrast media such as those containing indocyanine green, methylene blue, sodium fluorescein, and 5-aminolevulinic acid (5-ALA), and positron emission tomography (PET) contrast media such as those containing [¹⁸F]fluorodeoxyglucose (FDG) or [¹⁸F]sodium fluoride (Na¹⁸F).

In some embodiments the fluid injected by the actuator 50 herein may be in the form of a polymeric solutions, for example, solutions of a water-soluble polymer selected from polysaccharides (e.g., solutions of hyaluronic acid, gelatin, pectin, alginate, cellulose, gellan gum, etc.), polyethylene glycol, polyoxazolines, polypeptides, polyacrylates, polyacrylamides, copolymers of polyethylene glycol, and polyvinyl alcohol.

In some embodiments the fluid injected by the actuator 50 may be in the form of a shear thinning hydrogel, which is able to temporarily fluidize under shear stress and recover its original mechanical properties after release of the applied stress, based on natural polymers such as gelatin (e.g., Obsidio™ Conformable Embolic from Boston Scientific, a pre-hydrated bioresorbable mixture of gelatin and layered silicate particles, either with or without tantalum powder for radiocontrast), collagen, hyaluronic acid, alginate, and chitosan. Such hydrogels are generally crosslinked based on physical crosslinking mechanisms such as electrostatic interactions or hydrogen bonding.

In some embodiments the fluid injected by the actuator 50 may form crosslinked hydrogels in vivo. An example of such a crosslinked hydrogel is SpaceOAR®, which is based on a multi-arm polyethylene glycol (PEG) polymer functionalized with succinimidyl glutarate as activated end groups which further react with trilysine to form crosslinks. During use, a solution of the multi-arm polymer and the lysine is simultaneously injected with a buffer solution. When mixed, the buffer solution increases the pH and dramatically accelerates the rate of reaction between the multi-arm polymer and the lysine, forming a crosslinked hydrogel within seconds. Another example of such a crosslinked hydrogel is SpaceOAR Vue®, which, like SpaceOAR®, is based on a multi-arm polyethylene glycol (PEG) polymer functionalized with succinimidyl glutarate as activated end groups which further react with trilysine to form crosslinks. In SpaceOAR Vue®, some of the succinimidyl glutarate end groups are functionalized with 2,3,5-triiiodobenzamide groups, providing radiopacity. In other embodiments, systems of these types can be used to form hydrogels ex vivo, after which the hydrogels are broken down into particles and suspended in an aqueous solution to form an injectable pre-formed hydrogel.

In some embodiments the fluid injected by the actuator 50 may comprise a hydrophilic polymer hydrogel that comprises crosslinks between hydrophilic polymer chains within the hydrophilic polymer hydrogels, which crosslinks contain hydrolysable linkers, as described above.

In some embodiments the fluid injected by the actuator 50 may comprise a hydrophilic polymer hydrogel that comprises crosslinks between hydrophilic polymer chains within the hydrophilic polymer hydrogels, which crosslinks contain immolative linkers that are dispersed throughout the hydrogels, as described above. Such hydrogels can be degraded in situ by contact with a suitable cleavage composition, as described above.

The fluid injected by the actuator 50 for use in the present disclosure may also contain one or more additional agents such as therapeutic agents, imaging agents, colorants, tonicity adjusting agents, and pH adjusting agents. In embodiments where the balloon permits release (e.g., by allowing diffusion through the balloon), such agents (e.g., therapeutic agents, imaging agents, etc.) may be released from the balloon over time.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, mRNA, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g., green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of tonicity adjusting agents include sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others.

Examples of pH adjusting agents include various buffer solutes.

In other aspects, the present disclosure provides kits that include the actuator 50, one or more catheters and/or access sheaths, and one or more containers (e.g., syringes) that include a fluid to be injected by the actuator 50. Containers for the fluid or the fluid precursors include, for example, vials, ampules, empty syringes, and preloaded syringes.

In some embodiments, the kits herein include one or more containers that contain a cleavage solution. Containers for the cleavage solution include, for example, vials, ampules, and preloaded syringes. In these embodiments, the kits may further include a catheter tube for delivering the cleavage solution. In these embodiments, the kits may further include a needle for delivering a fluid (e.g., a hydrogel and/or a cleavage solution). In some embodiments, a distribution head, such as a spray head, may be disposed at a distal end of the catheter tube.

In some embodiments, the kits further include sterile packaging in which the above-described the kit components are removably packaged in a sterile state.

What is claimed is:

1. A hydrogel deployment actuator comprising:
a housing having an ergonomic hand grip;
a syringe tray configured to be movably disposed in the housing and configured to removably receive and secure a syringe;
a dual-axis swivel head assembly comprising:
a dual-axis swivel head;
a vertical swivel knob configured to move the dual-axis swivel head along a vertical plane;
a horizontal swivel knob configured to move the dual-axis swivel head along a horizontal plane;
a swivel lock mechanism configured to lock both vertical and horizontal movement of the dual-axis swivel head; and
a flexible tube configured to fluidically couple the dual-axis swivel head to a proximal end of the syringe; and
a mechanical trigger mechanism comprising:
a trigger lever pivotally mounted to the ergonomic hand grip; and
a swivel hammer coupled to the trigger lever, wherein the swivel hammer is configured to convert trigger lever movement into linear actuation force imparted on a plunger of the syringe.

2. The actuator of claim 1, wherein the vertical swivel knob and the horizontal swivel knob are positioned at a proximal end of the actuator.

3. The actuator of any claim 1, wherein the trigger lever is positioned on the ergonomic hand grip.

4. The actuator of claim 3, wherein the vertical swivel knob and the horizontal swivel knob are positioned on opposing vertical surfaces of the ergonomic hand grip.

5. The actuator of claim 1, wherein the swivel lock mechanism is configured to selectively lock and unlock both the horizontal swivel knob and the vertical swivel knob.

6. The actuator of claim 1, wherein the swivel lock mechanism is a slidable mechanism configured to translate between a locked position and an unlocked position.

7. The actuator of claim 1, wherein:
the swivel lock mechanism is an individual lock configured to simultaneously lock and unlock both the horizontal swivel knob and the vertical swivel knob; and
the swivel lock mechanism is located on the ergonomic hand grip at between the horizontal swivel knob and the vertical swivel knob.

8. The actuator of claim 1, wherein the syringe tray is configured to:
contact and maintain a body of the syringe in fixed alignment with the syringe tray;
contact a proximal end of a plunger of the syringe; and
permit metered longitudinal translation of a plunger of the syringe while maintaining the body of the syringe in the fixed alignment.

9. The actuator of claim 8, wherein the syringe tray includes a plurality of notches disposed along opposing substantially longitudinally extending surfaces of the syringe tray, and wherein each of the notches is configured to receive at least a portion of the swivel hammer.

10. The actuator of claim 9, wherein the housing is formed of a casing top rotatably coupled to a casing bottom, and wherein a proximal end of the casing bottom is rotatably coupled to a proximal end of the casing top.

11. The actuator of claim 10, wherein the housing includes a viewing window.

12. The actuator of claim 11, wherein the viewing window is located in a top surface of the housing and is aligned with the syringe tray such that at least a body of the syringe is visible through the viewing window when the syringe is loaded in the syringe tray.

13. The actuator of claim 1, further comprising an ultrasound probe attachment mechanism configured to rigidly mount an ultrasound probe to an exterior surface of the housing.

14. The actuator of claim 13, wherein the ultrasound probe attachment mechanism further comprises one or more projections configured to receive a flexible strap.

15. The actuator of claim 13, wherein the ultrasound probe attachment mechanism is located on a bottom surface of the housing.

16. A hydrogel deployment actuator comprising:
a housing having:
an ergonomic hand grip projecting at an angle from a proximal end of the housing; and
a cavity in the housing; and
an aperture extending through a distal end of the housing;
a syringe tray configured to be slidably disposed in the cavity of the housing and configured to removably receive and secure a syringe, wherein the syringe tray includes a plurality of notches disposed along at least a portion of a longitudinal length of the syringe tray;
a dual-axis swivel head assembly comprising:
a dual-axis swivel head located at a distal end of the housing, wherein a distal end of the dual-axis swivel head includes a luer end extending through the aperture at the distal end of the housing, wherein the proximal end of the luer end is in fluidic communication with a distal end of the syringe and the distal end of the luer end is configured to receive a needle;
a vertical swivel knob configured to move the dual-axis swivel head along a vertical plane, wherein the vertical swivel knob is located on the ergonomic hand grip and is coupled via a first drive wire to the dual-axis swivel head;
a horizontal swivel knob configured to move the dual-axis swivel head along a horizontal plane, wherein the horizontal swivel knob is located on the ergonomic hand grip and is coupled to the dual-axis swivel head via a second drive wire;
a swivel lock mechanism configured to simultaneously lock both vertical and horizontal movement of the dual-axis swivel head; and
a flexible tube configured to fluidically couple the dual-axis swivel head to a proximal end of the syringe;
a mechanical trigger mechanism comprising:
a trigger lever pivotally mounted to the housing; and
a swivel hammer coupled to the trigger lever, wherein:
the swivel hammer is configured to convert trigger lever movement into metered distal translation of the syringe tray to impart a metered linear actuation force on a plunger of the syringe by the syringe tray, and
the swivel hammer is disposable at least partially in a notch of the plurality of notches in the syringe tray.

17. The actuator of claim 16, further comprising an ultrasound probe attachment mechanism configured to rigidly mount an ultrasound probe to the actuator.

18. The actuator of claim 16, wherein:
the vertical swivel knob and the horizontal swivel knob are positioned on opposing vertical surfaces of the ergonomic hand grip; and
the swivel lock mechanism is a slidable mechanism located on the ergonomic hand grip that is configured to translate between:
a locked position to simultaneously lock drive wires coupled to both the vertical swivel knob and the horizontal swivel knob; and
an unlocked position to simultaneously unlock drive wires coupled to both the vertical swivel knob and the horizontal swivel knob.

19. A kit for delivering a hydrogel, the kit comprising:
a hydrogel deployment actuator comprising:
a housing having an ergonomic hand grip;
a syringe tray configured to be movably disposed in the housing and configured to removably receive and secure a syringe, wherein the syringe tray includes a plurality of notches disposed along at least a portion of a longitudinal length of the syringe tray;
a dual-axis swivel head assembly comprising:
a dual-axis swivel head;
a vertical swivel knob configured to move the dual-axis swivel head along a vertical plane, wherein the vertical swivel knob is coupled via a first drive wire to the dual-axis swivel head;
a horizontal swivel knob configured to move the dual-axis swivel head along a horizontal plane, wherein the horizontal swivel knob is coupled to the dual-axis swivel head via a second drive wire;
a swivel lock mechanism configured to simultaneously lock both vertical and horizontal movement of the dual-axis swivel head; and
a flexible tube configured to fluidically couple the dual-axis swivel head to a proximal end of the syringe;
a mechanical trigger mechanism comprising:
a trigger lever pivotally mounted to the housing; and
a swivel hammer coupled to the trigger lever, wherein:
the swivel hammer is configured to convert trigger lever movement into metered distal translation of the syringe tray to impart a metered linear actuation force on a plunger of the syringe by the syringe tray, and
the swivel hammer is disposable in a notch of the plurality of notches in the syringe tray; and
an ultrasound probe attachment mechanism configured to rigidly mount an ultrasound probe to the actuator; and
a syringe including an injectable hydrogel.

20. The kit of claim 19, wherein the syringe is an individual syringe, and wherein the syringe tray is configured to receive the individual syringe.
